(19) United States
(12) Patent Application Publication
(10) Pub. No.: US 2016/0379338 A1
(43) Pub. Date: Dec. 29, 2016

(54) REHABILITATION SUPPORTING INSTRUMENT AND REHABILITATION DEVICE

(71) Applicant: Seiko Epson Corporation, Tokyo (JP)

(72) Inventors: Yuya MARUYAMA, Chino (JP); Hideki TANAKA, Chino (JP); Takayuki KITAZAWA, Suwa (JP)

(21) Appl. No.: 15/184,131

(22) Filed: Jun. 16, 2016

(30) Foreign Application Priority Data
Jun. 29, 2015 (JP) 2015-129591

(51) Int. Cl. G06T 3/00; A61H 1/02; A61B 90/00; G06F 3/01

(52) U.S. Cl. CPC G06T 3/0006; G06F 3/014; A61H 1/0285; A61B 90/39

(57) ABSTRACT

A sheet for rehabilitation used together with a head-mounted display device (an HMD) that can display an image is disclosed. On the sheet for rehabilitation, markers functioning as labels for designating a position for displaying the image are provided.

### BACKGROUND

1. Technical Field

The present invention relates to a rehabilitation supporting instrument and a rehabilitation device.

2. Related Art

As a rehabilitation device, there has been known a device that shows a paralyzed body portion to a patient (a user) as if the paralyzed body portion is moving. For example, in a rehabilitation device described in JP-A-2015-39522 (Patent Literature 1), a marker is stuck to a paralyzed hand and, by using a head-mounted display device, a moving image serving as a model of a motion is displayed in a display position of the hand recognized by the marker.

JP-A-2015-103010 (Patent Literature 2) is also an example of related art.

In the rehabilitation device described in Patent Literature 1, the marker needs to be stuck to the paralyzed hand of the patient (the user). However, since the paralyzed hand is a disabled portion, it is not easy to attach the marker. It is likely that the marker prevents the movement of the hand and the patient (the user) cannot smoothly perform rehabilitation exercise. Besides, there have been demands for a reduction in the size, a reduction in costs, resource saving, facilitation of manufacturing, improvement of convenience of use, and the like of the device.

### SUMMARY

An advantage of some aspects of the invention is to solve at least a part of the problems described above, and the invention can be implemented as the following aspects.

(1) An aspect of the invention is directed to a rehabilitation supporting instrument used together with a body-mounted display device that can display an image. The rehabilitation supporting instrument includes a marker functioning as a label for designating a position where the image is displayed. With the rehabilitation supporting instrument according to this aspect, since it is unnecessary to attach a marker to a body portion of a patient (a user), it is possible to solve a problem of the attachment of the marker. Further, it is possible to prevent a situation in which rehabilitation exercise is not smoothly performed because of the marker.

(2) In the rehabilitation supporting instrument according to the aspect, the rehabilitation supporting instrument may further include a plane section on which a body portion of a user can be disposed, and the marker may be provided in the plane section. With the rehabilitation supporting instrument according to this aspect, in a head-mounted display device, it is possible to display an augmented reality image to be superimposed on the body portion disposed in the plane section. Therefore, it is possible to improve a rehabilitation effect by using the rehabilitation supporting instrument in this aspect.

(3) In the rehabilitation supporting instrument according to the aspect, the rehabilitation supporting instrument may include a plurality of the markers respectively corresponding to a plurality of parts of a body portion of a user. With the rehabilitation supporting instrument according to this aspect, in the head-mounted display device, it is possible to display the augmented reality image to be more accurately superimposed on the body portion.

(4) In the rehabilitation supporting instrument according to the aspect, the body portion may be a hand, and the markers may be associated with fingers of the hand. With the rehabilitation supporting instrument according to this aspect, it is possible to utilize the rehabilitation supporting instrument for rehabilitation of the hand.

(5) In the rehabilitation supporting instrument according to the aspect, an illustration image for showing a disposition position of a body portion of a user may be printed. With the rehabilitation supporting instrument according to this aspect, the user can easily learn in which position a disabled body portion is disposed. Therefore, convenience of use of the rehabilitation supporting instrument is high.

(6) In the rehabilitation supporting instrument according to the aspect, a disposition position of the marker may be able to be changed. With the rehabilitation supporting instrument according to this aspect, the disposition position of the marker can be changed according to the shape of a disabled body portion of the user. Therefore, it is possible to further improve reality of the augmented reality image.

(7) In the rehabilitation supporting instrument according to the aspect, the image may be a moving image showing a normal motion of a body portion of a user. With the rehabilitation supporting instrument according to this aspect, the user is capable of performing rehabilitation using a motion of the disabled body portion as a model. Therefore, it is possible to improve the rehabilitation effect.

(8) Another aspect of the invention is directed to a rehabilitation device for recovering a function of a disabled body portion. The rehabilitation device includes: a display device that can display an image; and a rehabilitation supporting instrument. In the rehabilitation supporting instrument, a marker associated with the body portion functioning as a label for designating a position where the image is displayed is provided in a position different from the body portion. With the rehabilitation device according to this aspect, like the rehabilitation supporting instrument in the aspect explained above, it is possible to solve the problem of the attachment of the marker. Further, it is possible to prevent a situation in which rehabilitation exercise is not smoothly performed because of the marker.

(9) In the rehabilitation device according to the aspect, the rehabilitation supporting instrument may include a plurality of the markers respectively corresponding to a plurality of parts of a body portion of a user, and the display device may include: a marker imaging section configured to image the markers; and a rehabilitation processing section configured to adjust the size and the display position of the image on the basis of the positions of the markers captured by the marker imaging section and perform the display. With the rehabilitation device according to this aspect, it is possible to appropriately adjust the size and the display position of the image.

(10) In the rehabilitation device according to the aspect, the rehabilitation processing section may deform the shape of the image on the basis of an inclination angle of the markers captured by the marker imaging section and perform the display of the image. With the rehabilitation device according to this aspect, it is possible to appropriately adjust the shape of the image.

### BRIEF DESCRIPTION OF THE DRAWINGS

The invention will be described with reference to the accompanying drawings, wherein like numbers reference like elements.

FIG. 1 is an explanatory diagram showing the overall configuration of a rehabilitation device according to a first embodiment.

FIG. 2 is an explanatory diagram showing the configuration of a display section for left eye in detail.

FIG. 3 is a block diagram functionally showing the configuration of a head-mounted display device (an HMD).

FIG. 4 is an explanatory diagram showing a form of use of a sheet for rehabilitation.

FIG. 5 is a plan view showing a sheet for rehabilitation exclusive for the right hand.

FIG. 6 is a flowchart for explaining rehabilitation processing.

FIG. 7 is an explanatory diagram showing an example of an exercise model.

FIG. 8 is an explanatory diagram showing an example of an image visually recognized by a user during reproduction.

FIG. 9 is an explanatory diagram showing a form of use of the HMD in a second modification of the first embodiment.

FIG. 10 is an explanatory diagram showing a sheet for rehabilitation visually recognized by the user and the periphery of the sheet for rehabilitation.

FIG. 11 is an explanatory diagram showing one frame of a moving image reproduced in the second modification.

FIG. 12 is an explanatory diagram showing a plate for rehabilitation according to a second embodiment.

### DESCRIPTION OF EXEMPLARY EMBODIMENTS

Embodiments of the invention are explained below.

### A. First Embodiment

### A-1. Overall Configuration

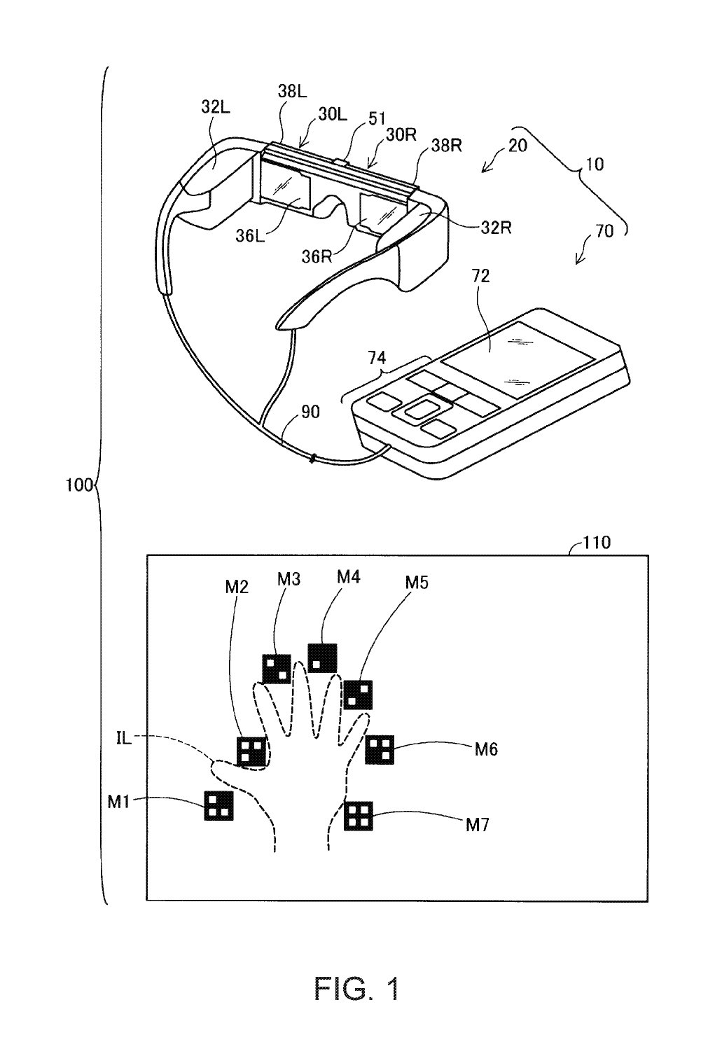

FIG. 1 is an explanatory diagram showing the overall configuration of a rehabilitation device 100 according to a first embodiment of the invention. As shown in the figure, the rehabilitation device 100 includes a head-mounted display device 10 and a sheet for rehabilitation 110. The rehabilitation device 100 is, as an example, a device for supporting bending and stretching of joints of a hand of a body to thereby recover functions of the hand. First, the head-mounted display device 10 is explained.

### A-2. Configuration of the Head-Mounted Display Device

The head-mounted display device 10 is a display device mounted on ahead and is also called head mounted display (HMD). The HMD 10 is an optical transmission type (a see-through type) with which a user can visually recognize a virtual image and, at the same time, visually recognize a real space.

The HMD 10 includes a display device 20 having a shape like eyeglasses and a control device (a controller) 70. The display device 20 and the control device 70 are communicably connected by wire or radio. In this embodiment, the display device 20 and the control device 70 are connected by a wired cable 90. The control device 70 communicates a signal of an image (an image signal) and a signal of control (a control signal) to and from the display device 20 via the cable 90.

The display device 20 includes a display section for the left eye (a display section for left eye) 30L and a display section for the right eye (a display section for right eye) 30R.

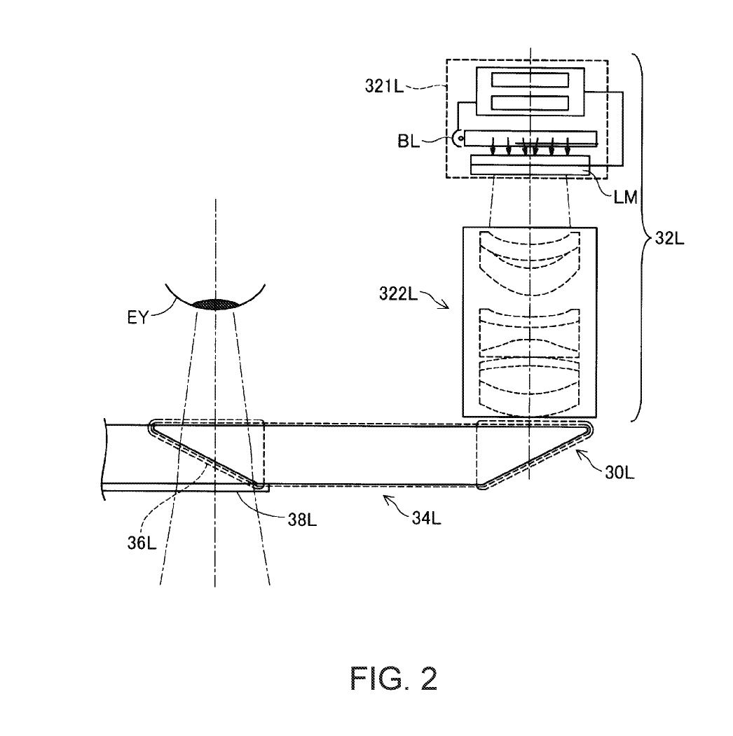

The display section for left eye 30L includes an image forming section for the left eye (an image forming section for left eye) 32L, a light guide section for the left eye (a light guide section for left eye 34L shown in FIG. 2), a reflecting section for the left eye (a reflecting section for left eye) 36L, and a shade for left eye 38L. The display section for right eye 30R includes an image forming section for the right eye (an image forming section for right eye) 32R, a light guide section for the right eye (same as the light guide section for left eye 34L shown in FIG. 2), a reflecting section for the right eye (a reflecting section for right eye) 36R, and a shade for right eye 38R.

FIG. 2 is an explanatory diagram showing the configuration of the display section for left eye 30L in detail. FIG. 2 is a view of the display section for left eye 30L viewed from right above. The image forming section for left eye 32L included in the display section for left eye 30L is disposed in a base portion of a temple of eyeglasses. The image forming section for left eye 32L includes an image generating section for the left eye (an image generating section for left eye) 321L and a projection optical system for the left eye (a projection optical system for left eye) 322L.

The image generating section for left eye 321L includes a light source of a backlight for the left eye (a backlight light source for left eye) BL and a light modulating element for the left eye (a light modulating element for left eye) LM. In this embodiment, the backlight light source for left eye BL includes a set of light sources for respective light emission colors such as red, green, and blue. As the light sources, for example, light emitting diodes (LEDs) and the like can be used. In this embodiment, the light modulating element LM includes a liquid crystal display device, which is a display element.

The display section for left eye 30L acts as explained below. When an image signal for the left eye is input to the image generating section for left eye 321L from the control device 70 (FIG. 1), the light sources of the backlight light source for left eye BL emit red light, green light, and blue light. The red light, the green light, and the blue light emitted from the light sources diffuse to be projected on the light modulating element for left eye LM. The light modulating element for left eye LM spatially modulates the projected red light, green light, and blue light according to the image signal input to the image generating section for left eye 321L from the control device 70 to thereby emit image light corresponding to the image signal.

The projection optical system for left eye 322L includes, for example, a projection lens group. The projection optical system for left eye 322L projects image light emitted from the light modulating element for left eye LM of the image generating section for left eye 321L and changes the image light to light beams of a parallel state. The image light changed to the light beams of the parallel state by the projection optical system for left eye 322L is projected on the light guide section for left eye 34L.

The light guide section for left eye 34L guides the image light from the projection optical system for left eye 322L to a predetermined surface (a semi-transmission reflection surface) of a triangular prism included in the reflecting section for left eye 36L. The front or the back of the semi-transmission reflection surface, which is formed in the reflecting section for left eye 36L, facing a left eye EY of the user during wearing is applied with reflection coating such as a mirror layer. The image light guided to the semi-transmission reflection surface formed in the reflecting section for left eye 36L is totally reflected toward the left eye EY of the user by the surface applied with the reflection coating. Consequently, image light corresponding to the guided image light is output from an area (an image extraction area) in a predetermined position of the reflecting section for left eye 36L. The output image light enters the left eye EY of the user and forms an image (a virtual image) on the retina of the left eye EY.

At least a part of light made incident on the reflecting section for left eye 36L from the real space is transmitted through the semi-transmission reflection surface formed in the reflecting section for left eye 36L and guided to the left eye EY of the user. Consequently, for the user, an image formed by the image forming section for left eye 32L and an optical image from the real space are seen as being superimposed.

The shade for left eye 38L is disposed on the opposite side of the left eye EY of the user in the light guide section for left eye 34L. In this embodiment, the shade for left eye 38L is detachable. The shade for left eye 38L is attached in a bright place or attached when the user desires to concentrate on a screen. Therefore, the user can clearly view the image formed by the image forming section for left eye 32L.

As shown in FIG. 1, the display section for right eye 30R includes a similar configuration symmetrical to the configuration of the display section for left eye 30L and acts in the same manner as the display section for left eye 30L. As a result, when the user wears the display device 20 on the head, for the user, an image corresponding to image light output from an image extraction area of the display device 20 (an image extraction area of the reflecting section for left eye 36L and an image extraction area of the reflecting section for right eye 36R) is seen as being displayed. Therefore, the user can recognize the image. At least a part of light from the real space is transmitted through the image extraction area of the display device 20 (the image extraction area of the reflecting section for left eye 36L and the image extraction area of the reflecting section for right eye 36R). Therefore, the user can view the real space while wearing the display device 20 on the head.

In this way, the user can simultaneously view (visually recognize) the image displayed on the image extraction area of the display device 20 (hereinafter simply referred to as “display image”) and the real space transmitted through the image extraction area. The display image serves as an AR image that gives augmented reality (AR) to the user.

In the display device 20, a camera 51 is provided in a position corresponding to the middle of the forehead of the user when the user wears the display device 20. Therefore, in a state in which the user wears the display device 20 on the head, the camera 51 picks up an image of the real space in a direction in which the user faces. The camera 51 is a monocular camera but may be a stereo camera.

The control device 70 is a device for controlling the display device 20. The control device 70 includes a touch pad 72 and an operation button section 74. The touch pad 72 detects contact operation on an operation surface of the touch pad 72 and outputs a signal corresponding to detection content. As the touch pad 72, various touch pads such as an electrostatic type, a pressure detection type, and an optical type can be adopted. The operation button section 74 includes various operation buttons, detects operation of the operation buttons, and outputs a signal corresponding to detection content. The touch pad 72 and the operation button section 74 are operated by the user.

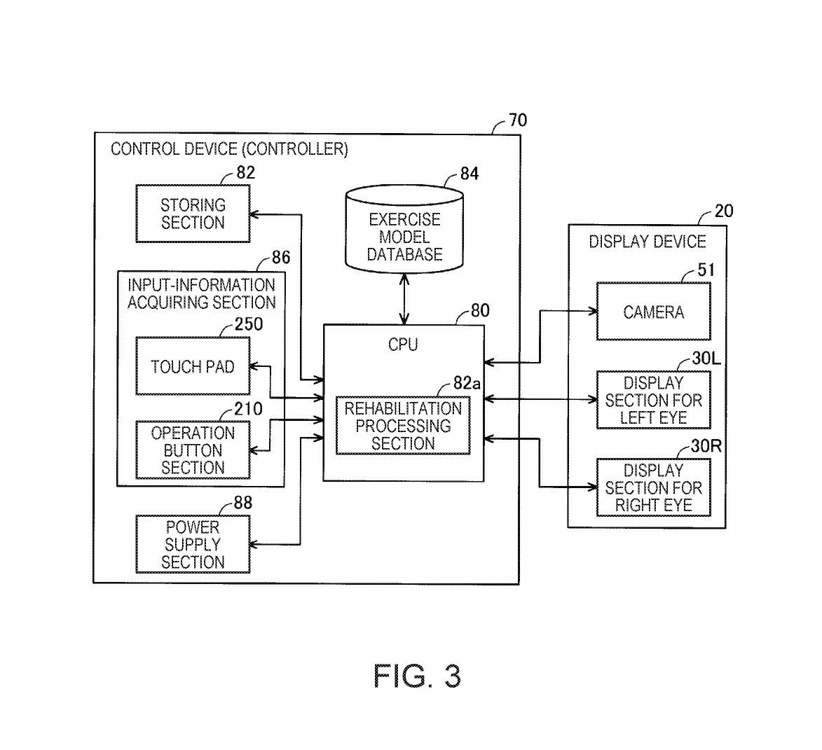

FIG. 3 is a block diagram functionally showing the configuration of the HMD 10. The control device 70 includes a CPU 80, a storing section 82, an exercise model database 84, an input-information acquiring section 86, and a power supply section 88. The sections are connected to one another by a bus or the like.

The storing section 82 includes a ROM, a RAM, a DRAM, or a hard disk. In the storing section 82, various computer programs such as an operating system (OS) are stored. In this embodiment, there is a computer program for rehabilitation as one of the stored computer programs.

The exercise model database 84 is a database in which exercise models are accumulated. The exercise model is moving image data obtained by modeling exercise set as a target in rehabilitation. In this embodiment, an exercise model for the left hand and an exercise model for the right hand are accumulated in advance. Note that the exercise model may be a collection of several still image data instead of the moving image data. Further, the exercise model may be data including a set of feature point positions of a hand. The exercise model can be replaced with any data as long as a moving image can be constructed from the data. Further, the exercise model may include parameters such as the number of times, speed, and the like of exercise.

The input-information acquiring section 86 includes the touch pad 72 and the operation button section 74. The input-information acquiring section 86 receives an input of a signal corresponding to the detection content received from the touch pad 72 or the operation button section 74.

The power supply section 88 supplies electric power to the components requiring the electric power in the control device 70 and the display device 20.

The CPU 80 reads out and executes the computer programs stored in the storing section 82 to thereby achieve various functions. Specifically, the CPU 80 achieves a function of executing, when detection content of operation is input from the input-information acquiring section 86, processing corresponding to the detection result, a function of reading data from and writing data in the storing section 82, and a function of controlling supply of electric power from the power supply section 88 to the components.

The CPU 80 reads out and executes the computer program for rehabilitation stored in the storing section 82 to thereby also function as a rehabilitation processing section 82a that executes rehabilitation processing for recovering functions of a disabled body portion. In this embodiment, the rehabilitation processing is processing for recovering functions of a hand, which is a disabled body portion. As a disability, there is, for example, a paralysis due to a stroke.

### A-3. Sheet for Rehabilitation

In FIG. 1, the sheet for rehabilitation 110 is indicated by a plan view. The sheet for rehabilitation 110 is a rectangular and thin sheet-like body. In this embodiment, the size of the sheet for rehabilitation 110 is 400 [mm]×600 [mm] and the thickness of the sheet for rehabilitation 110 is 3 [mm]. The size and the thickness are examples and do not need to be limited to these values. The size may be any size as long as at least one hand can be placed on the sheet for rehabilitation 110. In this embodiment, the sheet for rehabilitation 110 is made of synthetic resin. Note that the sheet for rehabilitation 110 may be made of another material such as thick paper instead of the synthetic resin.

An illustration image IL and first to seventh seven markers M1 to M7 are printed on the surface of the sheet for rehabilitation 110. The illustration image IL is an illustration-like image representing the palm side of the left hand. In this embodiment, the illustration image IL is an image of a state in which the joints of the fingers are stretched and the fingers are spread apart (a so-called “paper” state in rock-paper-scissors game). The size of the illustration image IL is an average size of the left hand of an adult. The surface of the sheet for rehabilitation 110 is a subordinate concept of the “plane section”.

The markers M1 to M7 are two-dimensional markers and serve as labels for designating a target object to which augmented reality is given. In this embodiment, the first to seventh markers M1 to M7 are printed around the contour of the left hand in the illustration image IL. Specifically, the first marker M1 is printed on the outer side of the thumb in the illustration image IL, the second to fifth markers M2 to M5 are printed respectively among five fingers from the thumb to the little finger in the illustration image IL, the sixth marker M6 is printed on the outer side of the little finger in the illustration image IL, and the seventh marker M7 is printed on one side (in this embodiment, the little finger side) of both the outer sides of the wrist portion.

Note that the positions of the markers M1 to M7 are positions suitable for specifying the outer edge of the hand and are not limited to the example explained above. For example, as a modification of this embodiment, the markers M1 to M7 can be set in various positions. For example, the seventh marker M7 can be printed on the thumb side of both the outer sides of the wrist portion. The number of markers is not limited to seven and can be set to various numbers. For example, the number of markers can be set to eight in total by providing markers on both of the little finger side and the thumb side of the wrist portion, can be set to six in total (the markers M1 to M6) excluding the marker in the wrist portion, can be set to four in total (the markers M2 to M5) only among the five fingers from the thumb to the little finger, and can be set to two in total (the markers M1 and M6) only on the outer sides of the thumb and the little finger. One marker can be printed instead of a plurality of markers. For example, only the marker M7 can be printed.

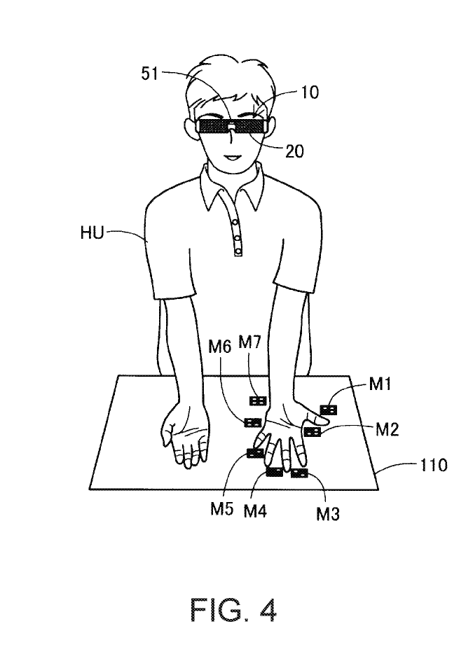

FIG. 4 is an explanatory diagram showing a form of use of the sheet for rehabilitation 110 in the configuration explained above. The sheet for rehabilitation 110 is placed on a not-shown desk, table, or the like and used. In a state in which a user (a patient) HU wears the display device 20 of the HMD 10, the user HU stretches out the disabled left hand over the sheet for rehabilitation 110. Specifically, the user HU places the left hand on the illustration image IL (FIG. 1) in a state in which the user HU turns the palm of the left hand upward and opens the hand. In this way, the user HU locates the thumb of the left hand between the first marker M1 and the second marker M2, places the index finger of the left hand between the second marker M2 and the third marker M3, places the middle finger of the left hand between the third marker M3 and the fourth marker M4, places the ring finger of the left hand between the fourth marker M4 and the fifth marker M5, places the little finger of the left hand between the fifth marker M5 and the sixth marker M6, and places the little finger side of both the outer sides of the wrist portion near the seventh marker M7.

As a result, the first to seventh markers M1 to M7 play a role of specifying the position of the hand placed on the surface of the sheet for rehabilitation 110. If a part of the markers M1 to M7 is hidden by the hand, it is seen that the shape of the hand or an opened state of the hand deviate from a standard model specified from the printed positions of the markers M1 to M7. In the state shown in FIG. 4, the user HU operates the touch pad 72 and the operation button section 74 (FIG. 1) of the control device 70 using the right hand to thereby instruct the HMD 10 to execute the rehabilitation processing. Note that, when the right hand of the user HU is also disabled, a person other than the user such as an assistant may operate the control device 70.

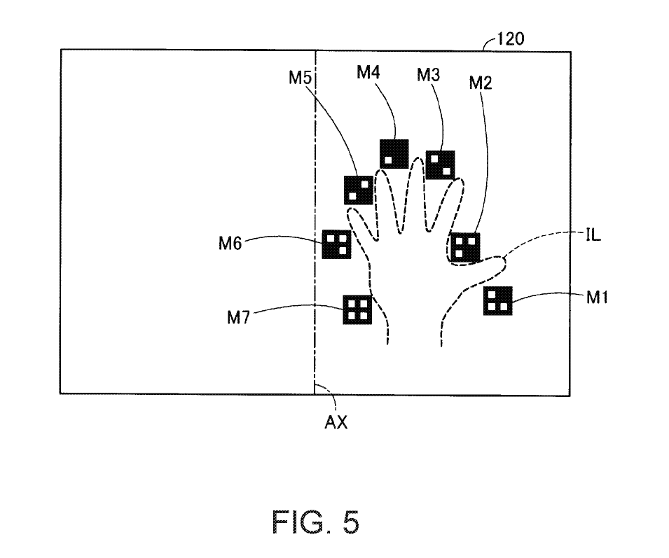

FIG. 5 is a plan view showing a sheet for rehabilitation 120 exclusive for the right hand. The sheet for rehabilitation 110 shown in FIG. 1 is used for the left hand and exclusive for the left hand as explained above. On the other hand, as shown in FIG. 5, the sheet for rehabilitation 120 exclusive for the right hand is a pattern symmetrical to the pattern of the sheet for rehabilitation 110 shown in FIG. 1 with respect to a perpendicular AX in the middle in the lateral direction set as a symmetrical axis. Note that patterns of the markers M1 to M7 are the same as the patterns of the sheet for rehabilitation 110 exclusive for the left hand except that disposition positions of the markers M1 to M7 are symmetrical to the disposition positions of the markers M1 to M7 of the sheet for rehabilitation 110 exclusive for the left hand. The sheets for rehabilitation 110 and 120 are subordinate concept of the “rehabilitation supporting instrument”.

### A-4. Rehabilitation Processing

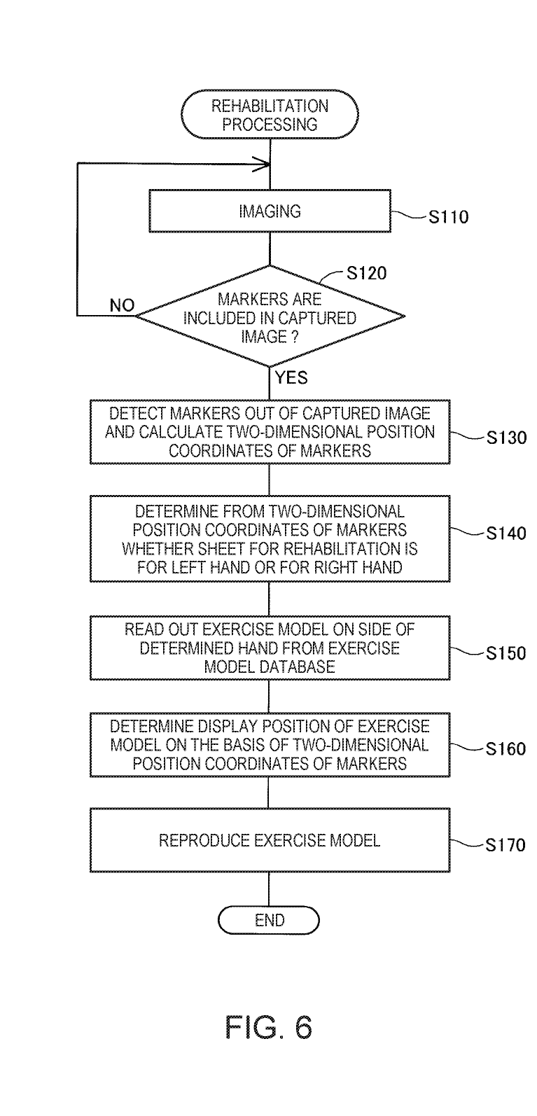

FIG. 6 is a flowchart for explaining the rehabilitation processing executed by the control device 70. The rehabilitation processing is processing by the rehabilitation processing section 82a (FIG. 3). When predetermined operation performed using the touch pad 72 and the operation button section 74 is received from the input-information acquiring section 86 (FIG. 3), execution of the rehabilitation processing is started by the CPU 80.

When the processing is started, first, the CPU 80 performs imaging with the camera 51 (step S110) and determines whether the markers M1 to M7 of the sheet for rehabilitation 110 are included in a captured image obtained by the imaging (step S120). “The markers M1 to M7 are included” means that all of the seven markers M1 to M7 are included. When at least one of the markers M1 to M7 is not included, it is determined that the markers M1 to M7 are not included.

In performing rehabilitation conforming to the rehabilitation processing, a user moves the eyes to the left hand, which is a body portion on which the rehabilitation is performed. The camera 51 picks up an image of a real space in a direction that the user faces. Therefore, when the user moves the eyes to the left hand, the markers M1 to M7 are included in a captured image by the camera 51. The determination in step S120 is affirmative. In the case of the affirmative determination, the CPU 80 advances the processing to step S130. On the other hand, if determining in step S120 that the markers M1 to M7 are not included, the CPU 80 returns the processing to step S110 and repeatedly executes the processing in steps S110 and S120.

In step S130, the CPU 80 detects the markers M1 to M7 out of the captured image obtained in step S110 and calculates two-dimensional position coordinates of the markers M1 to M7. A coordinate system indicating the two-dimensional position coordinates corresponds to a display screen by the display device 20. Subsequently, the CPU 80 determines from the calculated two-dimensional position coordinates of the markers M1 to M7 whether the sheet for rehabilitation 110 is for the right hand or for the left hand (step S140). Specifically, if determining from the two-dimensional position coordinates of the markers M1 to M7 that the first marker M1 is located on the left side with respect to the other markers M2 to M6 as shown in FIG. 1, the CPU 80 determines that the sheet for rehabilitation 110 is for the left hand. On the other hand, if determining that the first marker M1 is located on the right side with respect to the other markers M2 to M6, the CPU 80 determines that the sheet for rehabilitation 110 is for the right hand. Note that this method of determination is an example. The determination may be performed by any method as long as the determination is performed from a positional relation of the disposition of the markers M1 to M7.

Subsequently, the CPU 80 reads out, from the exercise model database 84, an exercise model on the side of the hand determined in step S140 (step S150). That is, if determining in step S140 that the sheet for rehabilitation 110 is for the left hand, the CPU 80 reads out an exercise model for the left hand. If determining in step S140 that the sheet for rehabilitation 110 is for the right hand, the CPU 80 reads out an exercise model for the right hand.

Subsequently, the CPU 80 determines, on the basis of the two-dimensional position coordinates of the markers M1 to M7 calculated in step S130, a region for displaying the exercise model (hereinafter referred to as “display region”) on the display surface of the display device 20 (step S160). The CPU 80 reproduces, in the determined display region, the exercise model read out in step S150 (step S170).

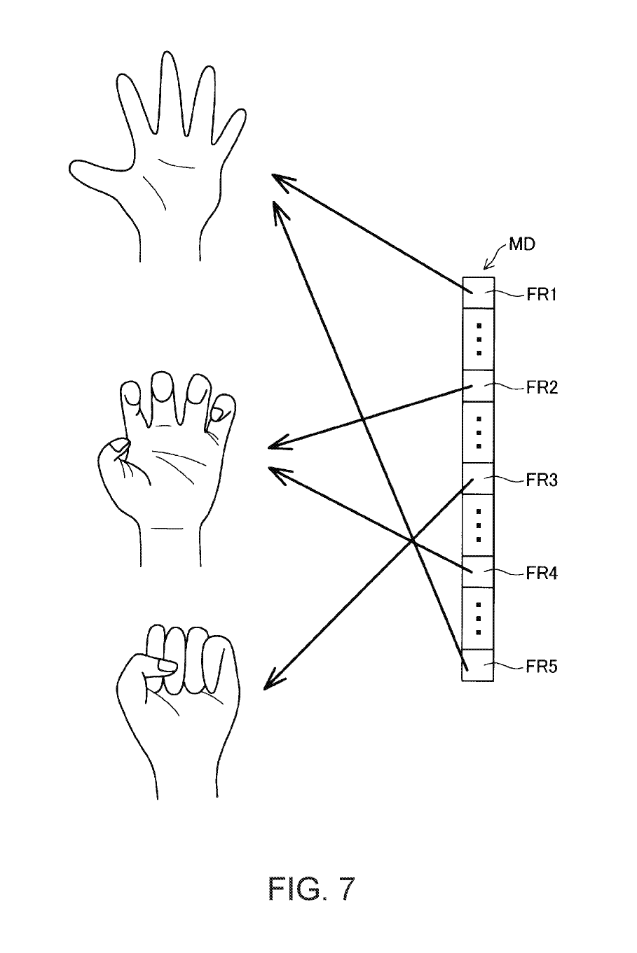

FIG. 7 is an explanatory diagram showing an example of the exercise model read out in step S150. An illustrated exercise model MD is an exercise model for the left hand. The exercise model MD is moving image data configured by a plurality of frames (still images) FR1, FR2, FR3, FR4, and FR5. One or a plurality of frames are included among the frames FR1 to FR5 as well.

The first frame FR1 represents a state in which a hand is opened (a so-called “paper” state in rock-paper-scissors game). The state in which the hand is opened coincides with the state of the hand represented as the illustration image IL in the sheet for rehabilitation 110. The frame FR3 in the center position of the plurality of frames represents a state in which the hand is closed (a so-called “rock” state in rock-paper-scissors game). The frame FR2 in the middle of the first frame FR1 and the center frame FR3 represents a state in which the joints of the fingers are bent halfway from the state in which the hand is opened, that is, a halfway state between the state in which the hand is opened and the state in which the hand is closed. The frame FR4 in the middle of the center frame FR3 and the last frame FR5 is the same as the frame FR2. The last frame FR5 is the same as the frame FR1.

With the exercise model MD configured as explained above, exercise for changing the state in which the hand is opened to the state in which the hand is closed and subsequently returning from the state in which the hand is closed to the state in which the hand is opened, that is, opening and closing exercise of the hand is indicated.

In step S160 in FIG. 6, the CPU 80 determines, as the display region, the position and the size of the hand to be displayed. Since the first to seventh markers M1 to M7 specify the outer edge of the hand, it is possible to decide the size of the hand to be displayed from the two-dimensional position coordinates of the markers M1 to M7. It is possible to decide the position of the hand to be displayed from one or a plurality of the markers M1 to M7. Note that, for the determination of the size, all of the first to seventh markers M1 to M7 may be used or two or more of the first to seventh markers M1 to M7 may be used.

It is also possible to determine the size and the position of the display region using only any one of the first to seventh markers M1 to M7. In this embodiment, since the first to seventh markers M1 to M7 are printed in advance, an assumed size of the hand is unconditionally decided. Moreover, it is possible to estimate the distance from the sheet for rehabilitation 110 to the HMD 10 by measuring a size in a picked-up image of one marker. Therefore, the size and the position of the display region may be determined using only one of the first to seventh markers M1 to M7.

In the subsequent step S170, the CPU 80 adjusts the size and the position of the exercise model such that the exercise model read out in step S150 matches the determined size and the determined position of the display region and then reproduces (displays) the exercise model in the display region. The display is performed by causing the display section for left eye 30L and the display section for right eye 30R explained above to operate. Note that, in adjusting the size and the position of the exercise model, the shape of the exercise model may be changed from picked-up images of the first to seventh markers M1 to M7. As explained above, if a part of the markers M1 to M7 is hidden by the hand, it is seen that the shape of the hand or an opened state of the hand deviate from a standard model specified from the printed positions of the markers M1 to M7. Therefore, the shape and the opened state of the hand may be estimated from the shape of the picked-up images of the markers M1 to M7. The exercise model may be matched with the estimated shape and the estimated opened state of the hand.

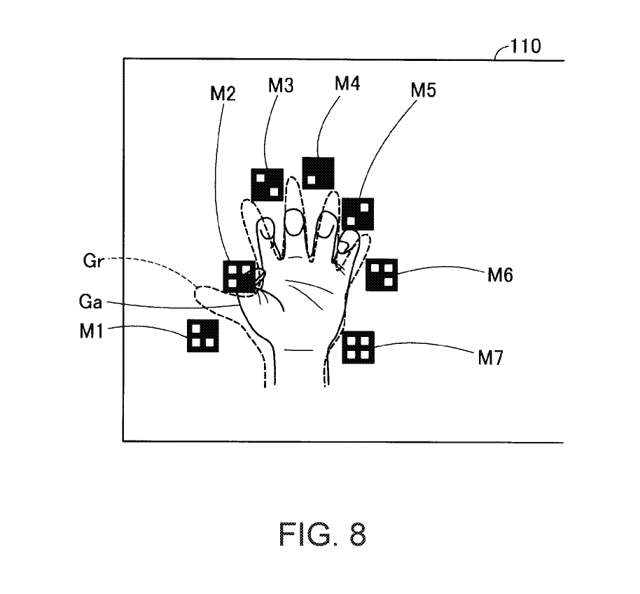

FIG. 8 is an explanatory diagram showing an example of an image visually recognized by the user during reproduction. An image of the hand indicated by a solid line in the figure is an image (an AR image) Ga of the reproduced exercise model. An image of the hand indicated by an alternate long and two short dashes line in the figure is an image (a real image) Gr of the hand of the user present in the real space seen through the sheet for rehabilitation 110. As shown in the figure, for the user, the image Ga of the exercise model is superimposed on the image Gr of the hand of the user and visually recognized. In the example shown in the figure, the image Ga of the exercise model is in a state in which the joints of the fingers are bent halfway. The image Gr of the hand of the user is in a state in which the hand is opened. The user performs rehabilitation exercise by opening and closing the hand following the movement of the image Ga of the exercise model from the state in which the hand is opened.

After the execution of step S170 in FIG. 6, the CPU 80 ends the rehabilitation processing once. Note that, in step S170, the CPU 80 performs the reproduction of the exercise model only once. However, instead, the CPU 80 may repeatedly reproduce the exercise model a plurality of times. Note that the user may be able to set the number of times of the repetition by operating the touch pad 72 and the operation button section 74 (FIG. 1) of the control device 70. Reproduction speed of the exercise model in step S170 is decided in advance. However, instead, the user may be able to set the reproduction speed.

### A-5. Effects of the Embodiment

In the sheet for rehabilitation 110 in the first embodiment configured as explained above, the markers M1 to M7 for specifying the position of the hand placed on the surface of the sheet for rehabilitation 110 are printed in advance on the surface of the sheet for rehabilitation 110. Therefore, since it is unnecessary to attach the markers M1 to M7 to a paralyzed hand, it is possible to solve a problem of the attachment of the markers M1 to M7 and prevent a situation in which the rehabilitation exercise is not smoothly performed because of the markers M1 to M7.

As explained above (see FIG. 8), the user can superimpose the image Ga of the exercise model on the image Gr of the hand of the user and visually recognize the image Ga of the exercise model. Therefore, the user can perform rehabilitation under an illusion that the image Ga of the exercise model is the hand of the user. Therefore, with the rehabilitation device 100 in this embodiment, it is possible to improve an effect of relieving a paralysis of a hand with the illusion effect.

### A-6. Modifications of the Embodiment

### First Modification

In the first embodiment, the illustration image IL is printed on the surface of the sheet for rehabilitation 110 together with the markers M1 to M7. On the other hand, as a first modification, a configuration may be adopted in which the illustration image IL is absent and only the markers M1 to M7 are printed. A method of placing the hand on the sheet for rehabilitation 110 is explained to the user (the patient) in advance. Then, the user can grasp a hand placement place only from the positions of the markers M1 to M7 and place the hand.

### Second Modification

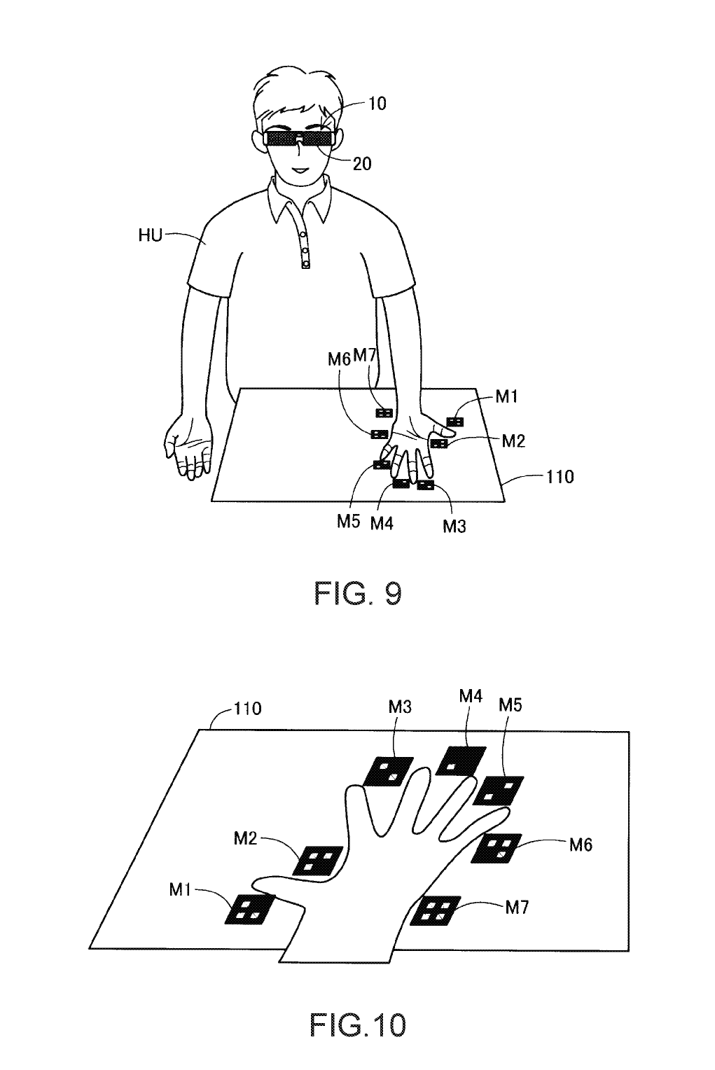

FIG. 9 is an explanatory diagram showing a form of use of the HMD in a second modification of the first embodiment. As shown in FIG. 9, depending on the position of the user, the position of the hand placed on the sheet for rehabilitation 110 is not the front but is an oblique lateral direction viewed from the user. In this case, the markers M1 to M7 and the hand in the real space seen through on the display screen of the HMD 10 are seen deformed as shown in FIG. 10. In the HMD in the second modification, it is determined whether images of the markers M1 to M7 detected out of a captured image by the camera 51 are rectangular. If the images are not rectangular, an angle of predetermined corner portions of the images is set as an inclination angle and deformation processing for deforming the exercise model on the basis of the inclination angle is performed. Thereafter, the exercise model after the deformation processing is reproduced.

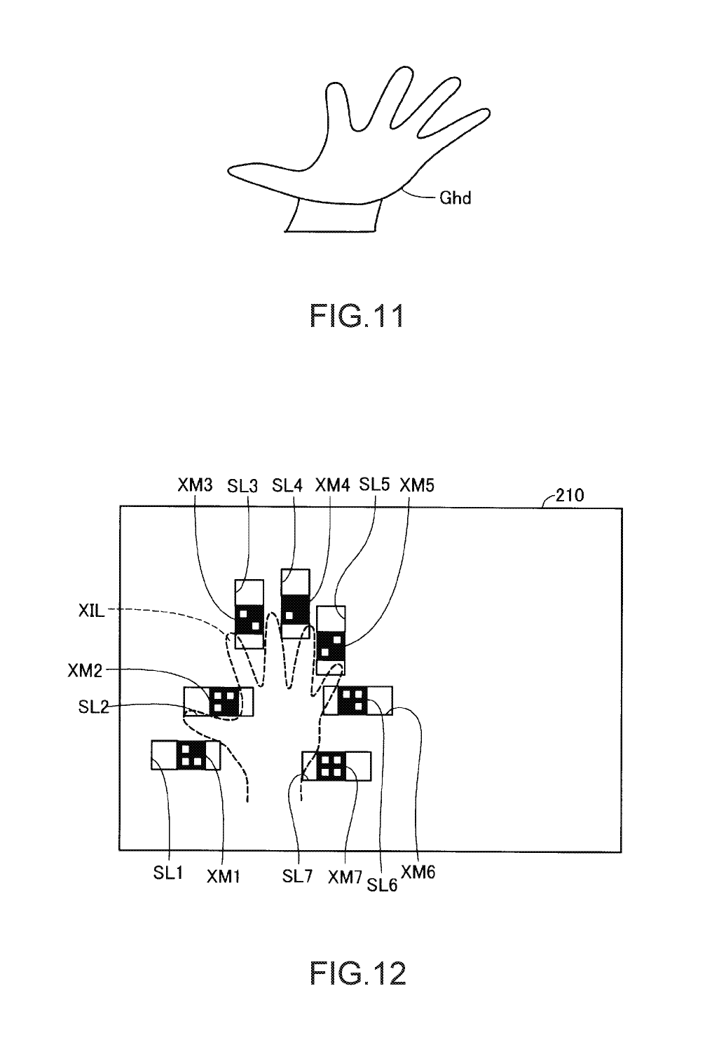

FIG. 11 is an explanatory diagram showing one frame of a moving image reproduced in the second modification. The moving image is a moving image by the exercise model after the deformation processing. An image Ghd representing the hand indicated by the one frame of the moving image is deformed like the image of the hand in the real space seen through on the display screen of the HMD 10. Therefore, the user can superimpose the image Ghd of the exercise model on the image of the hand of the user and visually recognize the image Ghd even from the oblique lateral direction. Therefore, according to the second modification, it is possible to further improve the illusion effect and further relieve the paralysis of the hand.

### Third Modification

The sheet for rehabilitation 110 in the first embodiment is the shin sheet-like body. On the other hand, as a third modification, the sheet for rehabilitation 110 may be a tabular plate for rehabilitation having thickness of, for example, 10 [mm] or more. The plate for rehabilitation is made of, for example, synthetic resin, metal, or wood. As in the first embodiment, an illustration image and markers are printed on the surface of the plate for rehabilitation. Note that, as in the first modification, the illustration image can be omitted. With this configuration, it is also possible to achieve an effect same as the effect of the first embodiment. A table for rehabilitation may be used instead of the sheet for rehabilitation and the plate for rehabilitation. The rehabilitation supporting instrument can be replaced with rehabilitation supporting instruments having various shapes.

### B. Second Embodiment

FIG. 12 is an explanatory diagram showing a plate for rehabilitation 210 according to a second embodiment of the invention. Like the sheet for rehabilitation 110 according to the first embodiment, the plate for rehabilitation 210 according to the second embodiment includes an illustration image XIL and first to seventh seven markers XM1 to XM7. As in the third modification of the first embodiment, the plate for rehabilitation 210 is made of synthetic resin, metal, or wood. The illustration image XIL is the same as the illustration image IL in the first embodiment. The markers M1 to M7 in the first embodiment are printed on the surface of the sheet for rehabilitation 110. The markers XM1 to XM7 in this embodiment include rectangular plate pieces. Images same as the markers M1 to M7 in the first embodiment are respectively printed on the surfaces of the plate pieces. The markers XM1 to XM7 are slidably disposed respectively in first to seventh slide grooves SL1 to SL7 formed on the plate for rehabilitation 210.

The first slide groove SL1 extends in an X direction in the figure (hereinafter referred to as left-right direction). The first marker XM1 disposed in the first slide groove SL1 is capable of sliding in the left-right direction. The position in the figure of the first marker XM1 is the same as the position of the first marker M1 in the sheet for rehabilitation 110 in the first embodiment (see FIG. 1). The first marker XM1 is manually slid in the left-right direction on the basis of the position.

The second slide groove SL2 extends in the left-right direction. The second marker XM2 disposed in the second slide groove SL2 is capable of sliding in the left-right direction. The position in the figure of the second marker XM2 is the same as the position of the second marker M2 in the sheet for rehabilitation 110 in the first embodiment. The second marker XM2 is manually slid in the left-right direction on the basis of the position.

The third slide groove SL3 extends in a Y direction in the figure (hereinafter referred to as up-down direction). The third marker XM3 disposed in the third slide groove SL3 is capable of sliding in the up-down direction. The position in the figure of the third marker XM3 is the same as the position of the third marker M3 in the sheet for rehabilitation 110 in the first embodiment. The third marker XM3 is manually slid in the up-down direction on the basis of the position.

The fourth slide groove SL4 extends in the up-down direction. The fourth marker XM4 disposed in the fourth slide groove SL4 is capable of sliding in the up-down direction. The position in the figure of the fourth marker XM4 is the same as the position of the fourth marker M4 in the sheet for rehabilitation 110 in the first embodiment. The fourth marker XM4 is manually slid in the up-down direction on the basis of the position.

The fifth slide groove SL5 extends in the up-down direction. The fifth marker XM5 disposed in the fifth slide groove SL5 is capable of sliding in the up-down direction. The position in the figure of the fifth marker XM5 is the same as the position of the marker M5 in the sheet for rehabilitation 110 in the first embodiment. The fifth marker XM5 is manually slid in the up-down direction on the basis of the position.

The sixth slide groove SL6 extends in the left-right direction. The sixth marker XM6 disposed in the sixth slide groove SL6 is capable of sliding in the left-right direction. The position in the figure of the sixth marker XM6 is the same as the position of the sixth marker M6 in the sheet for rehabilitation 110 in the first embodiment. The sixth marker XM6 is manually slid in the left-right direction on the basis of the position.

The seventh slide groove SL7 extends in the left-right direction. The seventh marker XM7 disposed in the seventh slide groove SL7 is capable of sliding in the left-right direction. The position in the figure of the seventh marker XM7 is the same as the position of the seventh marker M7 in the sheet for rehabilitation 110 in the first embodiment. The seventh marker XM7 is manually slid in the left-right direction on the basis of the position.

In a state in which the disabled left hand is placed on the plate for rehabilitation 210, the user slides the markers XM1 to XM7 with the normal right hand and disposes the markers XM1 to XM7 in contact with the outer edge of the left hand.

A rehabilitation device according to the second embodiment includes the sheet for rehabilitation and an HMD. The HMD includes components same as the components of the HMD 10 in the first embodiment. Further, the HMD changes the size and the shape of an exercise model on the basis of disposition positions of the markers XM1 to XM7 detected out of a captured image by the camera 51. Thereafter, the HMD reproduces the exercise model after the change.

In the plate for rehabilitation 210 in the second embodiment configured as explained above, as in the first embodiment, it is unnecessary to attach the markers XM1 to XM7 to the paralyzed hand. Therefore, it is possible to solve the problem of the attachment of the markers XM1 to XM7 and prevent a situation in which rehabilitation exercise is not smoothly performed because of the markers XM1 to XM7. With the rehabilitation device in the second embodiment, like the rehabilitation device in the first embodiment, it is possible to improve an effect of relieving a paralysis of a hand with the illusion effect in which the user misapprehends that an AR image of the exercise model is the hand of the user. In particular, in the plate for rehabilitation 210, it is possible to adjust the disposition positions of the markers M1 to M7 according to the size and the shape of the hand of the user. Therefore, it is possible to further match the size and the shape of the AR image to the hand of the user. Therefore, with the rehabilitation device in the second embodiment, it is possible to further improve the illusion effect and further improve the effect of relieving the paralysis of the hand.

Note that, in the plate for rehabilitation 210 in the second embodiment, the disposition positions of the markers XM1 to XM7 can be changed by sliding the slide grooves SL1 to SL7. However, the change of the disposition positions of the markers is not limited to this. The disposition positions of the markers may be changed by another method. As the other method, for example, the plate for rehabilitation may be a flat plate made of metal and permanent magnets may be provided in the markers or the plate for rehabilitation may be a cork board and pins may be provided in the markers. The plate for rehabilitation 210 is not limited to a plate shape and can also be a table for rehabilitation.

### C. Modifications

The invention is not limited to the first and second embodiments and the modifications thereof and can be carried out in various forms without departing from the spirit of the invention. For example, modifications explained below are also possible.

### Modification 1

In the embodiments and the modifications, the HMD is a transmission-type display device in which the visual field of the user is not blocked in the mounted state of the HMD. On the other hand, as a modification, the HMD may be a non-transmission-type display device in which the visual field of the user is blocked. In the non-transmission-type HMD, an image of the real space is captured by a camera and an AR image is superimposed on the captured image. In the embodiments and the modifications, the HMD includes the display section for left eye and the display section for right eye. However, the HMD may include only a display section for one eye instead of the display section for left eye and the display section for right eye.

### Modification 2

In the embodiments and the modifications, as the display device that can display the AR image, the head-mounted display device mounted on the head of the user is used. However, the display device is not limited to this. Various modifications of the display device are possible. For example, like a display device supported by an arm mounted on the shoulder or the neck of the user, a body-mounted display device mounted on the body of the user such as the head, the shoulder, or the neck may be used. The display device may be a display device of a placed type placed on a table or the like rather than being mounted on the user.

### Modification 3

In the embodiments and the modifications, the rehabilitation device is a device for recovering the functions of the hand of the patient. On the other hand, as a modification, the rehabilitation device may be a device for recovering the functions of the wrist and the arm joint. Further, the rehabilitation device may be a device for recovering the functions of the toes, the ankle, and the knee.

### Modification 4

In the embodiments and the modifications, the rehabilitation processing section 82a (FIG. 3) is explained as being realized by the CPU 80 executing the computer program stored in the storing section 82. However, the rehabilitation processing sect ion may be configured using an ASIC (Application Specific Integrated Circuit) designed to realize the function of the rehabilitation processing section.

### Modification 5

In the embodiments and the modifications, the camera 51 is integrally attached to the display device 20. However, the display device 20 and the camera 51 may be separately provided.

The invention is not limited to the embodiments, the examples, and the modifications and can be realized in various configurations without departing from the spirit of the invention. For example, the technical features in the embodiments, the examples, and the modifications corresponding to the technical features in the aspects described in the summary can be substituted or combined as appropriate in order to solve a part or all of the problems explained above or achieve a part of all of the effects explained above. Unless the technical features are explained as essential technical features in this specification, the technical features can be deleted as appropriate.

The entire disclosure of Japanese Patent Application No. 2015-129591 filed Jun. 29, 2015 is expressly incorporated by reference herein.

What is claimed is:

1. A rehabilitation supporting instrument used together with a body-mounted display device that can display an image, the rehabilitation supporting instrument comprising a marker functioning as a label for designating a posit ion where the image is displayed.

2. The rehabilitation supporting instrument according to claim 1, further comprising a plane section on which a body portion of a user can be disposed, wherein
the marker is provided in the plane section.

3. The rehabilitation supporting instrument according to claim 1, wherein the rehabilitation supporting instrument includes a plurality of the markers respectively corresponding to a plurality of parts of a body portion of a user.

4. The rehabilitation supporting instrument according to claim 3, wherein
the body portion is a hand, and
the markers are associated with fingers of the hand.

5. The rehabilitation supporting instrument according to claim 1, wherein an illustration image for showing a disposition position of a body portion of a user is printed.

6. The rehabilitation supporting instrument according to claim 1, wherein a disposition position of the marker can be changed.

7. The rehabilitation supporting instrument according to claim 1, wherein the image is a moving image showing a normal motion of a body portion of a user.

8. A rehabilitation device comprising:
a display device that can display an image; and
a rehabilitation supporting instrument, wherein
the rehabilitation supporting instrument includes a marker functioning as a label for designating a position where the image is displayed.

9. The rehabilitation device according to claim 8, wherein
the rehabilitation supporting instrument includes a plurality of the markers respectively corresponding to a plurality of parts of a body portion of a user, and
the display device includes:
a marker imaging section configured to image the markers; and
a rehabilitation processing section configured to adjust a size and a display position of the image on the basis of positions of the markers captured by the marker imaging section and perform the display of the image.

10. The rehabilitation device according to claim 9, wherein the rehabilitation processing section deforms a shape of the image on the basis of an inclination angle of the markers captured by the marker imaging section and performs the display of the image.
